(19) United States
(12) Patent Application Publication
(10) Pub. No.: US 2020/0029891 A1
(43) Pub. Date: Jan. 30, 2020

(54) VENIPUNCTURE AND ARTERIAL LINE GUIDANCE VIA SIGNAL VARIATION AMPLIFICATION

(71) Applicant: KONINKLIJKE PHILIPS N.V., EINDHOVEN (NL)

(72) Inventor: Christine Menking SWISHER, San Diego, CA (US)

(21) Appl. No.: 16/488,789

(22) PCT Filed: Feb. 21, 2018
(86) PCT No.: PCT/EP2018/054314

§ 371 Date: Aug. 26, 2019

Related U.S. Application Data
(60) Provisional application No. 62/463,895, filed on Feb. 27, 2017.

(51) Int. Cl. A61B 5/00

(52) U.S. Cl. CPC A61B 5/489; A61B 5/1535; A61B 5/0077; A61B 5/6898

(57) ABSTRACT

A vasculature imaging device includes an optical camera (10), a display (12), an electronic processor (14) connected to operate the optical camera and the display, and a non-transitory storage medium (16) storing instructions (18) readable and executable by the electronic processor to perform a vasculature imaging method (20). That method includes: operating the optical camera to acquire color video; computing a temporal variation of values of pixels of the color video; identifying pixels representing vasculature based on the temporal variation of the values of the pixels; and operating the display to present the color video with highlighting of the pixels representing vasculature. In some embodiments, the vasculature imaging device comprises a cellular telephone (cell phone) or other mobile device (22) with the camera and display being built-in components. The instructions may be an application (app) executable under a mobile operating system (24) run by the mobile device.

### FIELD

The following relates generally to the venipuncture arts, arterial line placement arts, nursing and patient care arts, hematology arts, and related arts.

### BACKGROUND

Venipuncture and arterial line placement provide access to a patient's venous and arterial blood systems, respectively. Venipuncture is used for tasks such as drawing blood for testing or blood donation, administering intravenous (IV) fluids, and the like. Venipuncture is a very common medical procedure: by some estimates around one billion venipuncture procedures are performed each year. Arterial lines are used for drawing arterial blood gas (ABG) samples, direct arterial blood pressure monitoring, and the like. Venipuncture and arterial line placement are commonly performed by nurses, doctors, and other medical professionals. Accurate initial placement of the hypodermic needle or IV needle in venipuncture greatly improves patient experience by minimizing skin penetrations that can lead to pain and potential pathways for infection, and avoids delays and improves clinical workflow. However, by some estimates accurate placement on the first attempt is achieved less than half the time. Arterial line placement is a more difficult procedure due to the deeper location of arteries compared with veins, leading to increased pain and potential for injury in the case of repeated arterial line placement attempts.

The following discloses a new and improved systems and methods that address the above referenced issues, and others.

### SUMMARY

In one disclosed aspect, a vasculature imaging device includes an optical camera, a display, at least one electronic processor, and a non-transitory storage medium storing instructions readable and executable by the at least one electronic processor to perform a vasculature imaging method. That method includes: operating the optical camera to acquire color video; computing a temporal variation of values of pixels of the color video; identifying pixels representing vasculature based on the temporal variation of the values of the pixels; and operating the display to present the color video with highlighting of the pixels representing vasculature. In some embodiments, the vasculature imaging device comprises a cellular telephone (“cell phone”) or other mobile device with the camera and display with built-in components. The instructions may be an application (“app”) executable under a mobile operating system run by the mobile device.

In another disclosed aspect, a non-transitory storage medium stores instructions readable and executable by a mobile device running a mobile operating system and having a built-in display and a built-in an optical camera with color video acquisition capability. The instructions include an application executable under the mobile operating system run by the mobile device to perform a vasculature imaging method. That method includes the operations of (i) acquiring color video using the built-in optical camera of the mobile device; (ii) identifying pixels representing vasculature in the color video; and (iii) presenting the color video with highlighting of the pixels representing vasculature on the built-in display of the mobile device.

In another disclosed aspect, a vasculature imaging method is disclosed, which comprises: acquiring color video using an optical camera; performing electronic processing of the color video using an electronic processor, the electronic processing including computing a temporal variation of values of pixels of the color video and identifying pixels representing vasculature based on the temporal variation of the values of the pixels; and presenting the color video with highlighting of the pixels representing vasculature on a display. In some embodiments, the identifying includes differentiating between pixels representing veins and pixels representing arteries, and the presenting includes highlighting the pixels representing veins and the pixels representing arteries using different highlighting for the veins and the arteries.

One advantage resides in providing a vascular imaging device or method which improves the likelihood of successful first placement for venipuncture or arterial line placement procedures.

Another advantage resides in providing a vascular imaging device or method effective for improving success rates for venipuncture or arterial line placement procedures.

Another advantage resides in providing a vascular imaging device or method effective for distinguishing between arteries and veins.

Another advantage resides in providing vascular imaging device or method having one or more of the foregoing advantages which utilizes the camera, display, and electronic processor of an existing cellular telephone (cell phone) or other mobile device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a front view (upper left) and a back view (lower left) of a vasculature imaging device, along with a diagrammatic representation of internal electronics (upper right) and a vasculature imaging process performed by the vasculature imaging device (lower right).

FIG. 2 diagrammatically illustrates use of the vasculature imaging device of FIG. 1 for imaging vasculature in a target location for venipuncture or arterial line placement, e.g. a patient's wrist, hand, or lower arm region.

FIGS. 3 and 4 diagrammatically illustrates image processing operations of the vasculature imaging process of FIG. 2.

FIG. 5 presents a table of illustrative options for the color video acquisition, temporal variation computation, vascular pixel identification, and vasculature highlighting operations of the vascular imaging process shown in FIG. 1 (lower right).

### DETAILED DESCRIPTION

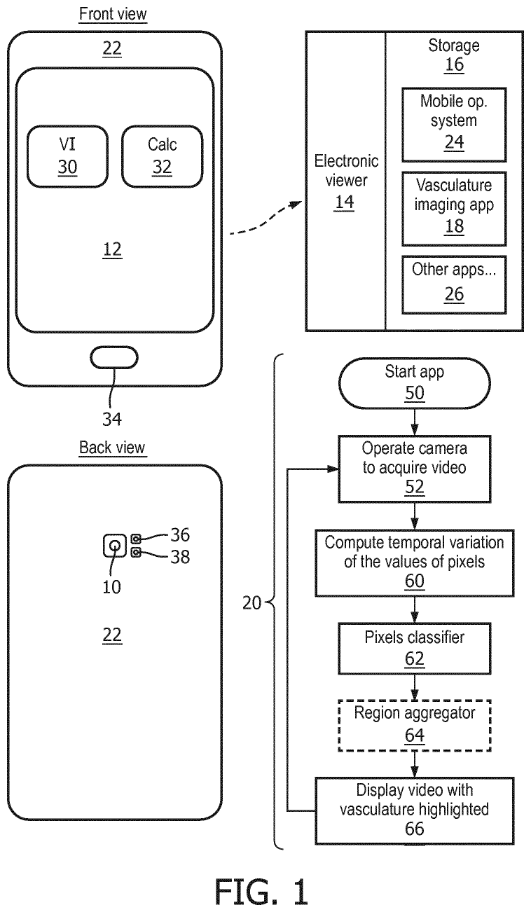

With reference to FIG. 1, an illustrative vasculature imaging device includes an optical camera 10 having color video acquisition capability, a display 12, and a diagrammatically indicated electronic processor 14 connected to operate the optical camera 10 and the display 12, along with a non-transitory storage medium 16 storing instructions 18 readable and executable by the electronic processor 14 to perform a vasculature imaging method 20 (diagrammatically indicated in FIG. 1 by way of a flow chart). The illustrative vasculature imaging device is implemented as a mobile device, e.g. an illustrative cellular telephone 22 (or in other embodiments, a tablet computer, personal data assistant or PDA, or so forth), in which the optical camera 10 is a built-in camera of the mobile device 22 and the display 12 is a built-in display of the mobile device 22. The illustrative mobile device 22 runs a mobile operating system 24, such as an iOS™ or Android™ operating system (available respectively from Apple Corp., Cupertino, Calif., U.S.A.; and Google Inc., Mountain View, Calif., U.S.A.) which is capable of running various applications (“apps”) 26 to cause the mobile device 22 to perform various tasks as programmed by the respective apps. FIG. 1 diagrammatically illustrates the mobile device-based vasculature imaging device by depicting a front view (upper left) and a back view (lower left) of the mobile device (e.g. cell phone) 22, along with a diagrammatic representation of internal electronics including the electronic processor 14 and non-transitory storage medium 16 (upper right) and the vasculature imaging process 20 performed by the vasculature imaging device (lower right). As is known in the art, the various apps 18, 26 including the vasculature imaging app 18 are stored locally in (or on) the non-transitory storage medium 16 of the mobile device 22, and are also typically stored at the non-transitory storage medium (e.g. hard drive, RAID, solid state drive, optical disk, or the like) of a network server that is accessible via a wireless communication network to which the mobile device 22 is connected via Wi-Fi, 4G, or another wireless communication link. In some commercial embodiments, a user downloads the app 18 from the server via the wireless communication network, either for free or after payment of a purchase fee or license purchase fee. It is additionally or alternatively contemplated for various apps 18, 26 to be loaded into the non-transitory storage medium 16 via a wired connection such as a USB cable. The non-transitory storage medium 16 of the mobile device 22 may, for example, be a flash memory, CMOS memory, or the like; while the electronic processor 14 may be a microcontroller or microprocessor which may be multi-core, and/or include a graphical processing unit (GPU), or be otherwise equipped to provide a desired level of computing power.

The display 12 may be an LCD display, an OLED display, or the like. The display 12 may have touch-sensitive overlay, e.g. employing capacitive or surface acoustic wave (SAW) touchscreen technology. The touch-sensitive display 12 thus serves as a user input device. In a typical design, the various apps 18, 26 have corresponding application icons, e.g. an icon 30 corresponding to the vascular imaging app 18, an illustrative icon 32 corresponding to a calculator app, and so forth. In response to detection, via the touch-sensitive overlay of the display 12, of a user touching the icon 30, the vascular imaging application 18 is loaded and starts executing. The mobile device 22 may include other user input controls, such as an illustrative “home” button 34.

The optical camera 10 typically includes a lens or lens assembly that forms an image on a digital detector array (e.g. a CCD imaging array, a CMOS imaging array, et cetera). The optical camera 10 has color video capability, e.g. by having an imaging array with pixels sensitive to red, green, and blue light (or another set of colors substantially spanning the visible spectrum, e.g. 400-700 nm). The optical camera 10 produces video frames comprising visible light images, i.e. with image content predominantly in the visible spectrum (400-700 nm), although some contribution(s) from the neighboring near-infrared and/or near-ultraviolet spectral region(s) is contemplated. The frame rate may, for example, be 24 frames/sec or 30 frames/sec, as non-limiting illustrative examples. Typically, the frame rate should be at least twice the highest frequency temporal variation that is expected to be analyzed, in order to satisfy the Nyquist sampling-rate criterion. As a heart rate of 300 beats per minute (5 beats/sec) is higher than physically realizable for most persons, a frame rate of at least 10 fps is expected to be sufficient to capture variations cycling with the heart rate. The optical camera 10 optionally may include other features, such as a built-in flash 36 and/or an ambient light sensor 38 for setting exposure times.

The illustrative vascular imaging device advantageously utilizes the built-in camera 10, built-in display 12, and built-in electronic processor 14 of the cellular telephone (cell phone) or other mobile device 22, thereby leveraging hardware already available to most nurses, doctors, and other medical professionals. In other contemplated embodiments, the vascular imaging device may be a dedicated device, e.g. including a dedicated optical camera with video capability mounted on a bracket or housing to hold the camera in fixed position which includes the subject's potential insertion site in the field of view (FOV) for venipuncture or arterial line placement. In another embodiment, a bracket including a cell phone holder is provided to hold the cell phone 22 in a convenient position for viewing the vasculature during the venipuncture or arterial line placement procedure.

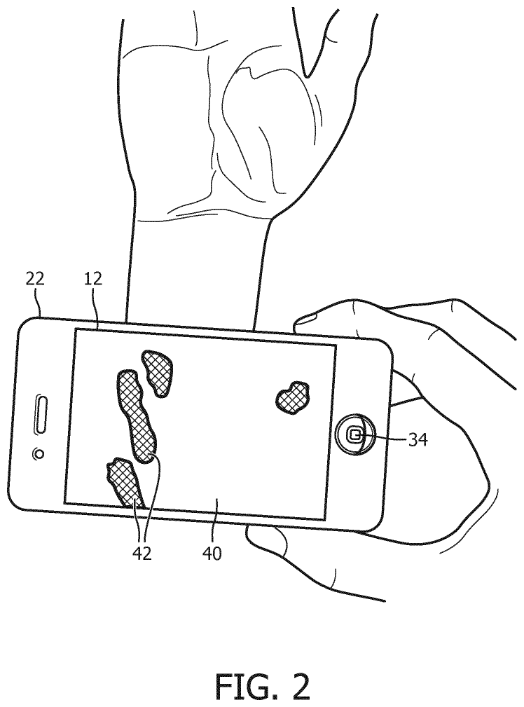

With brief reference to FIG. 2, in another approach the cell phone 22 is held by hand during the vascular imaging. As shown in FIG. 2, acquired color video is presented as a video display 40 shown on the display 12 of the cell phone 22. As described elsewhere herein, the color video is processed by the electronic processor 14 running the vasculature imaging application 18 to identify pixels representing vasculature, for example based on the temporal variation of the values of the pixels, and the color video is presented with highlighting 42 of the pixels representing vasculature. The highlighting 42 may, for example, include one or more of displaying the pixels representing vasculature in a specific color, displaying the pixels representing vasculature at a higher intensity than pixels not representing vasculature, and/or displaying the pixels representing vasculature with a time-varying intensity. In some embodiments, the processing differentiates between pixels representing venous vasculature and pixels representing arterial vasculature, and the veins and arteries are highlighted with different highlighting, e.g. a red color for veins and a blue color for arteries in one highlighting scheme.

With reference back to FIG. 1, the illustrative vasculature imaging method 20 is described in further detail. In an operation 50, the vasculature imaging method is initiated. For example, the operation 50 may entail the mobile operating system 24 running on the mobile device 22 detecting, via the touch-sensitive overlay of the display 12, a user touching the icon 30 and in response loading and executing the vascular imaging application 18. In an operation 52, the optical camera 10 is operated to acquire color video. (It is assumed here that the camera is pointed to image the arm, wrist, hand, or other body part at which the venipuncture or arterial line placement procedure is to be performed). In an operation 60, a temporal variation of the values of pixels of the color video is computed. As the color video includes a sequence of frames (i.e. image sequence) acquired over time, the temporal variation corresponds to variation in the pixel value over successive frames of the color video. Various temporal variations are expected to be particularly indicative of vasculature. For example, venous regions are expected to undergo color variation over time due to changes in venous blood oxygenation. As another example, arterial regions are expected to undergo variations due to subtle motion caused by influx and outflow of arterial blood. In an operation 62, pixels are classified as vasculature or non-vasculature (and optionally the vascular pixels are further differentiated into venous or arterial pixels) on the basis of the temporal variations. In an optional operation 64, a connectivity analysis or other grouping operation is used to group contiguous pixels identified as vasculature to delineate regions of vasculature. (In some variant embodiments, such connectivity analysis or grouping of pixels 64 may be performed before the pixel classification 62 or as an integral part of the pixel classification 62, e.g. in a region-growing pixel classification approach). In an operation 66, the color video acquired in operation 52 is displayed on the display 12, with the pixels identified as vasculature highlighted. Flow then passes back to operation 52. More particularly, in some embodiments interleaved processing is performed, e.g. in which the last N frames are being processed in operations 60, 62, 64 while the next N frames are being acquired via operation 52.

In the illustrative examples, the vasculature imaging process 20 is performed by the vascular imaging application 18 running on the electronic processor 14 of the mobile device 22. However, it is contemplated for portions or all of the computational operations, e.g. one or more, or all, of operations 60, 62, 64, to be performed by another electronic processor, such as at an Internet server such as a cloud computing resource. In these embodiments, the video captured by the operation 52 performed by the electronic processor 14 is transmitted via WiFi, cellular connection, or other wireless communication link to the external server or other second electronic processor which then executes the operations 60, 62, 64 to generate vasculature highlighting that is then transmitted back from the server to the mobile device 22 via the WiFi, cellular or other wireless communication link for display at the mobile device 22 via the operation 66 performed by the electronic processor 14 of the mobile device 22. Such a variant approach might be advantageous, for example, if the mobile device 22 has a fast wireless communication link but limited on-board processing power.

In the following some illustrative examples of more specific embodiments of the vasculature imaging process 20 are described. In some of these illustrative examples, an algorithm which amplifies the strength of signal variation is used to amplify subtle changes in color or motion within the color video. Regions with variations consistent with vasculature are identified and highlighted.

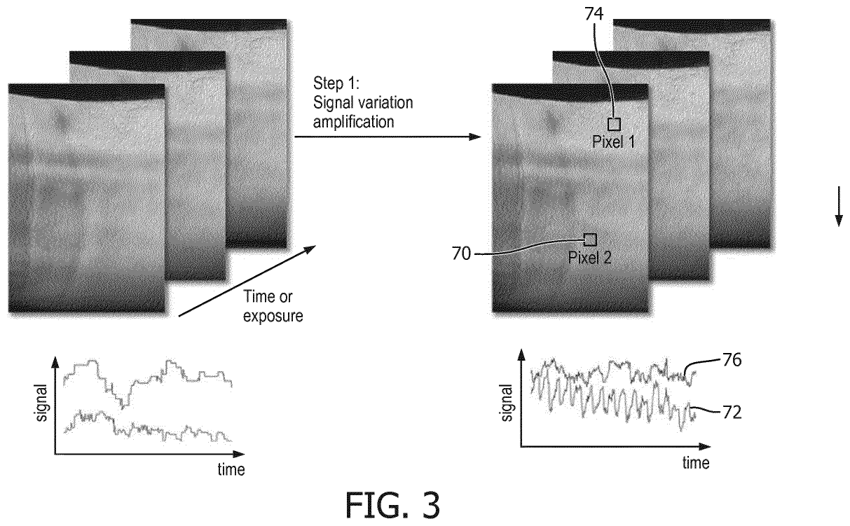

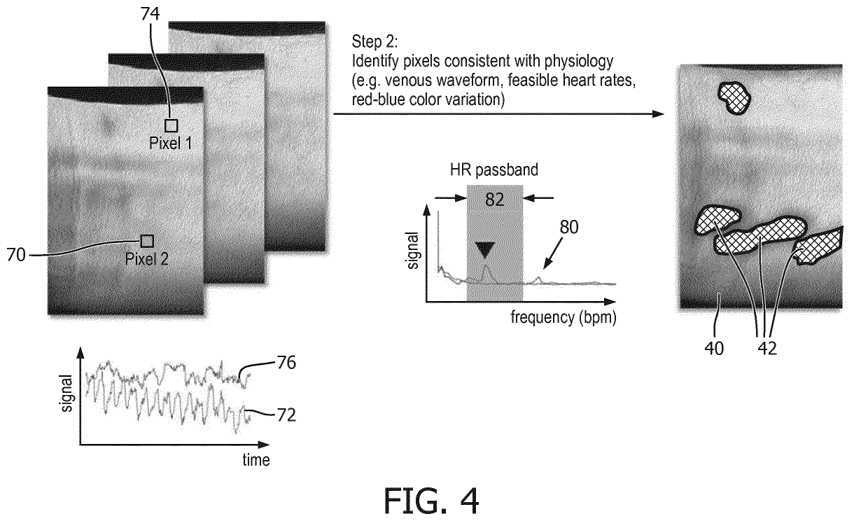

With continuing reference to FIG. 1 and with further reference to FIGS. 3 and 4, to detect and localize vasculature, the two-step approach of the vasculature imaging process 20 of FIG. 1 includes the first step 60, in which subtle signal variations in the color video are amplified. FIG. 3 illustrates one approach. In the example of FIG. 3, the operation 60 employs amplification of motion variation as using the Eulerian Motion Magnification algorithm. See Eulerian Video Magnification. Wu et al., “Eulerian Video Magnification for Revealing Subtle Changes in the World”, ACM Transactions on Graphics vol. 31 no. 4 (Proc. SIGGRAPH, 2012). The amplification of variations by this approach enhances variations not easily detected from the raw video. For example, as shown in FIG. 3, after signal amplification a pixel 70 which is over vasculature has a sinusoidal signal 72 in the expected frequency range consistent with the arterial waveform (e.g. corresponding the cardiac cycle or pulse rate). Another pixel 74, which is not located over vasculature, has a signal 76 that does not have a frequency consistent with physiology. With reference to FIG. 4, in the second step 62, pixels consistent with vascular physiology are identified. In the illustrative example of FIG. 4, the signal is decomposed into its frequency components via Fourier transform to produce frequency spectra 80. One method to extract information is to identify the peaks within the physiological feasible passband 82 (e.g. corresponding to the credible range of pulse rates for the patient, e.g. having a lower limit of 40 beats/min or some other lowest value that is realistic for a patient and an upper limit of 200 beats/min or some other highest value that is realistic for a patient) by windowing followed by a slope inversion and a local peak search. Other approaches can be employed to identify pixels representing vasculature based on the temporal variation of the values of the pixels produced by the computation 60.

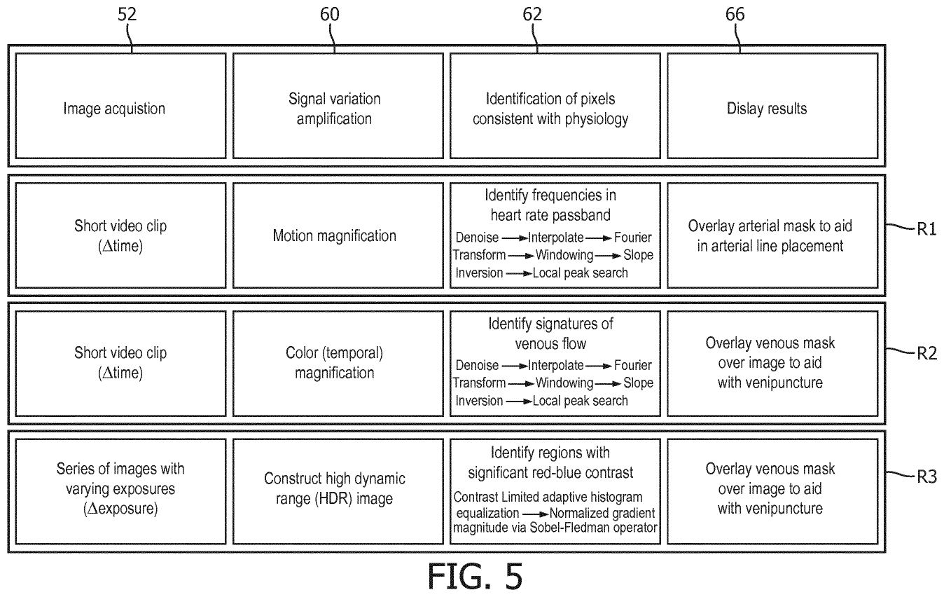

With reference to FIG. 5, numerous variants of the processing diagrammatically depicted in FIGS. 3 and 4 are contemplated, some of which are presented in FIG. 5, which presents a table of options for the color video acquisition operation 52, the temporal variation computation operation 60, the vascular pixel identification operation 62, and the vasculature highlighting component of the display operation 66. For instance, the addition of color variation amplification is expected to yield further improvements and remove sensitivity to gross motion. Additional signal processing steps such as interpolation, de-noising, and smoothing are expected to further improve accuracy. Sensitivity to color variations could additionally or alternatively be enhanced with High Dynamic Range (HDR) image acquisition. By extending the bit depth, small signal variations in color or brightness will have increased signal-to-noise (SNR). This is contemplated to be followed by adaptive histogram equalization algorithms to enhance contrast and/or directly followed by color variation amplification. In illustrative FIG. 5, row R1 provides processing expected to be especially useful in highlighting pixels representing arterial vasculature, while rows R2 and R3 are expected to be especially useful in highlighting pixels representing venous vasculature.

As noted in FIG. 1, in further variants it is contemplated to employ a region aggregator operation 64 to group together pixels representing vasculature into regions, either after the pixel classification (as shown in FIG. 1) or prior to or integrally with the pixel classification. For example, the region aggregator operation 64 may identify an isolated pixel that is identified in operation 62 as not representing vasculature but which is surrounded mostly or entirely by pixels that are identified in operation 62 as representing vasculature in this case the isolated pixel is identified in operation 64 as also representing vasculature. Conversely, an isolated pixel that is identified in operation 62 as representing vasculature but which is surrounded mostly or entirely by pixels that are identified in operation 62 as not representing vasculature is suitably identified in operation 64 as also not representing vasculature.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A vasculature imaging device comprising:
an optical camera having color video acquisition capability;
a display;
at least one electronic processor; and
a non-transitory storage medium storing instructions readable and executable by the at least one electronic processor to perform a vasculature imaging method including:
operating the optical camera to acquire color video;
computing a temporal variation of values of pixels of the color video;
identifying pixels, the identifying including at least identifying pixels representing vasculature based on a frequency component of the temporal variation of the values of the pixels corresponding to a credible range of pulse rates and
operating the display to present the color video with highlighting of the pixels representing vasculature.

2. The vasculature imaging device of claim 1 wherein:
the computing includes computing a temporal variation of color values of the pixels of the color video; and
the identifying includes identifying pixels representing vasculature based on the temporal variation of the color values of the pixels.

3. The vasculature imaging device of claim 1 wherein:
the computing includes computing an Eulerian video magnification of variation of values of the pixels of the color video; and
the identifying includes identifying pixels representing vasculature using the Eulerian video magnification.

4. (canceled)

5. The vasculature imaging device of claim 1 wherein the highlighting comprises one or more of displaying the pixels representing vasculature in a specific color, displaying the pixels representing vasculature at a higher intensity than pixels not representing vasculature, and displaying the pixels representing vasculature with a time-varying intensity.

6. The vasculature imaging device of claim 1 wherein the computing and the identifying comprises:
computing a first temporal variation of values of pixels of the color video and a second temporal variation of values of pixels of the color video wherein the second temporal variation is different from the first temporal variation;
identifying pixels representing veins based on the first temporal variation of the values of the pixels;
identifying pixels representing arteries based on the second temporal variation of the values of the pixels;
wherein the highlighting comprises displaying the pixels representing veins using a venous highlighting and displaying the pixels representing arteries using an arterial highlighting that is different from the venous highlighting.

7. The vasculature imaging device of claim 6 wherein:
the venous highlighting includes displaying the pixels representing veins using a red color highlighting; and
the arterial highlighting includes displaying the pixels representing arteries using a blue color highlighting.

8. The vasculature imaging device of claim 6 wherein:
the first temporal variation comprises a temporal variation of color values of the pixels of the color video; and
the second temporal variation comprises an Eulerian video magnification of variation of values of the pixels of the color video.

9. The vasculature imaging device of claim 1 wherein the vasculature imaging device comprises a mobile device and wherein the optical camera is a built-in camera of the mobile device and the display is a built-in display of the mobile device.

10. The vasculature imaging device of claim 9 wherein the mobile device is a cellular telephone (cellphone), the optical camera is a built-in camera of the cellphone and the display is a built-in display of the cellphone.

11. A non-transitory storage medium storing instructions readable and executable by a mobile device running a mobile operating system and having a built-in display and a built-in an optical camera with color video acquisition capability, the instructions including an application executable under the mobile operating system run by the mobile device to perform a vasculature imaging method including the operations of (i) acquiring color video using the built-in optical camera of the mobile device; (ii) identifying pixels representing vasculature in the color video by at least identifying pixels representing vasculature based on a frequency-based analysis of the temporal variation of the values of the pixels; and (iii) presenting the color video with highlighting of the pixels representing vasculature on the built-in display of the mobile device.

12. The non-transitory storage medium of claim 11 wherein the identifying operation (ii) includes (ii)(a) computing a temporal variation of values of pixels of the color video and (ii)(b) identifying the pixels representing vasculature based on the temporal variation of the values of the pixels.

13. The non-transitory storage medium of claim 12 wherein:
the computing operation (ii)(a) includes computing a temporal variation of values of the pixels of the color video; and
the identifying operation (ii)(b) includes identifying pixels representing veins based on the temporal variation of the color values of the pixels.

14. The non-transitory storage medium of claim 12 wherein:
the computing operation (ii)(a) includes computing a temporal variation of values of the pixels of the color video; and
the identifying operation (ii)(b) includes identifying pixels representing arteries using the Eulerian video magnification.

15. (canceled)

16. The non-transitory storage medium of claim 11 wherein the highlighting comprises one or more of displaying the pixels representing vasculature in a specific color, displaying the pixels representing vasculature at a higher intensity than pixels not representing vasculature, and displaying the pixels representing vasculature with a time-varying intensity.

17. The non-transitory storage medium of claim 11 wherein the mobile operating system is an iOS™ or Android™ operating system.

18. (canceled)

19. (canceled)

20. (canceled)
